(19) United States
(12) Patent Application Publication
(10) Pub. No.: US 2014/0081137 A1
(43) Pub. Date: Mar. 20, 2014

(54) ULTRASOUND DIAGNOSTIC APPARATUS AND METHOD FOR DISPLAYING AN ULTRASOUND IMAGE

(71) Applicant: GE Medical Systems Global Technology Company, LLC, Waukesha, WI (US)

(72) Inventor: Shunichiro Tanigawa, Tokyo (JP)

(73) Assignee: GE MEDICAL SYSTEMS GLOBAL TECHNOLOGY COMPANY, LLC, Waukesha, WI (US)

(21) Appl. No.: 14/029,212

(22) Filed: Sep. 17, 2013

(30) Foreign Application Priority Data
Sep. 18, 2012 (JP) 2012-204257

(51) Int. Cl. A61B 8/08

(52) U.S. Cl. CPC A61B 8/485
USPC 600/438

(57) ABSTRACT

An ultrasound diagnostic apparatus is provided. The ultrasound diagnostic apparatus includes a physical quantity calculating unit configured to set correlation windows to two echo signals different in time from each other on the same sound ray, and perform a correlation computation to calculate a physical quantity related to elasticity. The ultrasound diagnostic apparatus further includes a computing unit configured to compute a computed value from a computational equation, the computed value emphasizing parameter characteristics of a cyst in a biological tissue, the computational equation using at least two of the following three parameters: an absolute value of the physical quantity, a correlation coefficient related to the correlation computation, and an intensity of each echo signal of ultrasound obtained from the biological tissue. The ultrasound diagnostic apparatus further includes a display unit configured to display an image having a display form corresponding to the computed value.

### CROSS REFERENCE TO RELATED APPLICATIONS

This application claims the benefit of Japanese Patent Application No. 2012-204257 filed Sep. 18, 2012, which is hereby incorporated by reference in its entirety.

### BACKGROUND OF THE INVENTION

The present invention relates to an ultrasound diagnostic apparatus which displays an image based on echo signals obtained by transmission/reception of ultrasound to and from a biological tissue, and a control program thereof.

There has been known an ultrasound diagnostic apparatus, which combines a normal B-mode image and an elastic image indicative of the hardness or softness of a biological tissue together and displays the result of combination. In this type of ultrasound diagnostic apparatus, the elastic image is generated in the following manner, for example. First, the transmission/reception of ultrasound is performed on a biological tissue while deforming the biological tissue by, for example, repeating pressure by an ultrasound probe and its relaxation, thereby acquiring echoes. Then, a correlation computation is performed based on data about the resultant echoes to calculate a physical quantity related to the elasticity of the biological tissue. The physical quantity is converted to color information to generate a colored elastic image. Incidentally, for example, strain of the biological tissue or the like is calculated as the physical quantity related to the elasticity of the biological tissue. A method of calculating the strain has been disclosed in, for example, Japanese Unexamined Patent Publication No. 2008-126079.

Meanwhile, there is a case where a liquid portion called cyst exists in a biological tissue. In relation to the entirety of the cyst or its part, the value of strain calculated by the method of Japanese Unexamined Patent Publication No. 2008-126079 may take on a value indicating that the biological tissue is hard. Here, in an elastic image, the value of strain becomes a value indicating that a tumor represented as a lump is also hard as with the cyst. There was therefore a case where it was difficult to distinguish between the tumor and the cyst in the elastic image.

### BRIEF DESCRIPTION OF THE INVENTION

The systems and methods described herein utilize that in a cyst, by performing a correlation computation, each of values random in sign may be obtained as a physical quantity related to the elasticity of a biological tissue, As an absolute of those quantities, a value indicating that the biological tissue is softer than each portion other than the cyst is obtained, and the cyst has parameter characteristics small in correlation coefficient related to the correlation computation and small in the intensity of each echo signal. The systems and methods described herein include an ultrasound diagnostic apparatus which displays an image on which parameter characteristics seen in a cyst are indicated. Described specifically, there is proposed an ultrasound diagnostic apparatus including a physical quantity calculating unit which sets correlation windows to two echo signals different in time from each other on the same sound ray, the echo signals being obtained by transmission/reception of ultrasound to and from a biological tissue. The ultrasound diagnostic apparatus performs a correlation computation between the correlation windows to thereby calculate a physical quantity related to elasticity of each portion in the biological tissue. A computing unit computes a computed value of a computational equation from which a computed value emphasized in parameter characteristics of a cyst in the biological tissue and distinguishable from other than the cyst is obtained, the computational equation using at least two of the following three parameters: an absolute value of the physical quantity calculated by the physical quantity calculating unit, a correlation coefficient related to the correlation computation, and an intensity of each echo signal of ultrasound obtained from the biological tissue. A display unit displays an image having a display form corresponding to the computed value of the computing unit thereon.

According to above aspect, the ultrasound diagnostic apparatus is provided with a computing unit which computes a computed value of a computational equation from which a computed value emphasized in parameter characteristics of a cyst in the biological tissue is obtained, the computational equation using at least two parameters of three parameters that are an absolute value of a physical quantity calculated by the physical quantity calculating unit, a correlation coefficient related to the correlation computation, and an intensity of each echo signal of ultrasound obtained from the biological tissue. Since each image having a display form corresponding to the computed value of the computing unit is displayed, images capable of distinguishing between a tumor and the cyst can be displayed.

Further advantages will be apparent from the following description of exemplary embodiments as illustrated in the accompanying drawings.

### BRIEF DESCRIPTION OF THE DRAWINGS

FIG. 1 is a block diagram showing a schematic configuration of an ultrasound diagnostic apparatus according to a first embodiment.

FIG. 2 is a block diagram illustrating a configuration of a display controller in the ultrasound diagnostic apparatus shown in FIG. 1.

FIG. 3 is a diagram depicting a display unit on which a composite ultrasound image in which a B-mode image and a color image are combined together, is displayed.

FIG. 4 is a block diagram showing a schematic configuration of an ultrasound diagnostic apparatus according to a second embodiment.

FIG. 5 is a block diagram illustrating a configuration of a display controller in the ultrasound diagnostic apparatus shown in FIG. 4.

FIG. 6 is a diagram showing a display unit on which a composite ultrasound image in which a B-mode image and an elastic image are combined together, is displayed.

FIG. 7 is a diagram showing the display unit on which a composite ultrasound image in which a B-mode image and a color image are combined together, and a composite ultrasound image in which a B-mode image and an elastic image are combined together, are displayed.

### DETAILED DESCRIPTION OF THE INVENTION

Exemplary embodiments will hereinafter be described in detail based on the accompanying drawings.

### First Embodiment

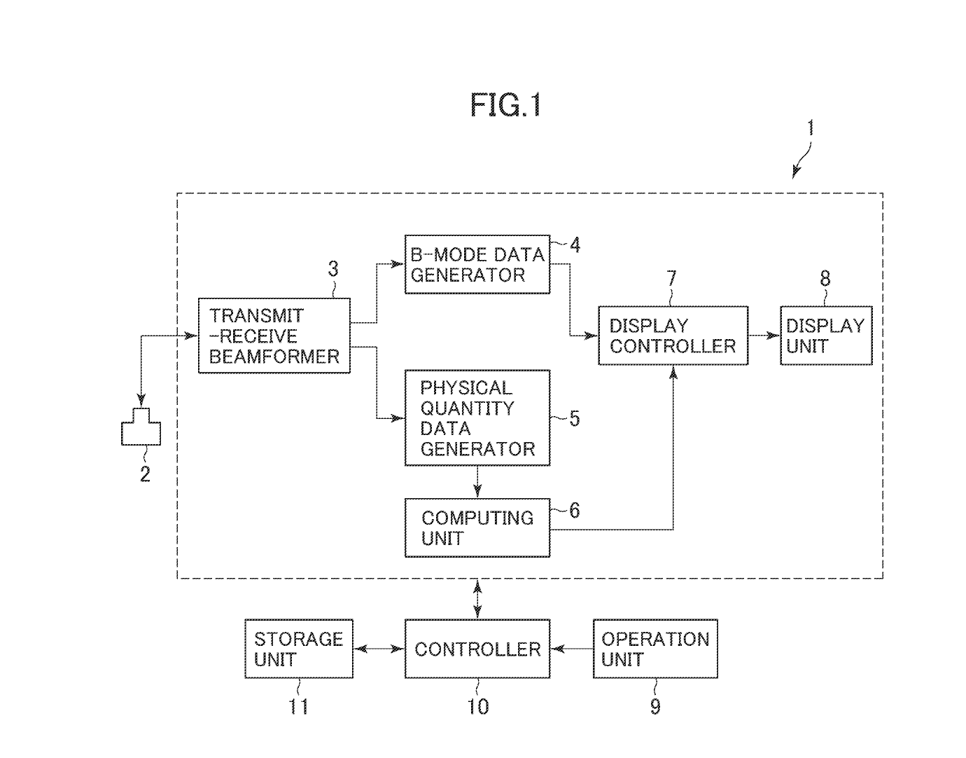

A first embodiment will first be explained based on FIGS. 1 through 3. An ultrasound diagnostic apparatus 1 shown in FIG. 1 is equipped with an ultrasound probe 2, a transmit-receive beamformer 3, a B-mode data generator 4, a physical quantity data generator 5, a computing unit 6, a display controller 7, a display unit 8, an operation unit 9, a controller 10 and a storage unit 11.

The ultrasound probe 2 transmits ultrasound to a biological tissue and receives its echoes. A B-mode image and a color image are generated as will be described later, based on echo data acquired by performing the transmission/reception of the ultrasound while deforming the biological tissue by repeating pressure and relaxation in a state in which the ultrasound probe 2 is being brought into contact with the surface of the biological tissue.

The transmit-receive beamformer 3 drives the ultrasound probe 2 under a predetermined scan condition, based on a control signal outputted from the controller 10 to perform the scanning of the ultrasound for every sound ray. The transmit-receive beamformer 3 performs signal processing such as phasing-adding processing on each echo received by the ultrasound probe 2. Echo data subjected to the signal processing by the transmit-receive beamformer 3 is outputted to the B-mode data generator 4 and the physical quantity data generator 5.

The B-mode data generator 4 performs B-mode processing such as logarithmic compression processing, envelop detection processing or the like on the echo data outputted from the transmit-receive beamformer 3 to thereby generate B-mode data. The B-mode data is outputted from the B-mode data generator 4 to the display controller 7.

The physical quantity data generator 5 generates data (physical quantity data) about a physical quantity related to the elasticity of each portion in the biological tissue, based on the echo data outputted from the transmit-receive beamformer 3 (physical quantity calculating function). As described in, for example, the above Japanese Unexamined Patent Publication No. 2008-126079, the physical quantity data generator 5 sets correlation windows to echo data different in time on the same sound ray at one scanning surface. The physical quantity data generator 5 computes a complex cross-correlation imaginary part between the correlation windows to calculate a physical quantity related to the elasticity, thereby generating the physical quantity data. More specifically, the physical quantity data generator determines compression rates of the waveforms of echo signals before and after (before and after pressure and before and after relaxation) deformation of the biological tissue by the ultrasound probe 2. The compression rate of each waveform described herein is a compression rate of a waveform between correlation windows. As a result, strain is obtained as the physical quantity related to the elasticity. The physical quantity data generator 5 sets a plurality of correlation windows in a sound ray direction and calculates strain related to each correlation window. Accordingly, the strains are calculated at a plurality of points (portions corresponding to correlation windows) on one sound ray.

Incidentally, each calculated strain is accompanied by a sign corresponding to the direction of displacement of a biological tissue. When pressure is performed by the ultrasound probe and its relaxation is performed, signs become opposite to each other. At a given time, however, results of computation obtained at respective portions (points) should be the same sign. For example, each portion should be a positive sign upon pressure, whereas upon relaxation, each portion should be a negative sign. In a cyst, however, the intensity of each echo signal is very low as described above. Thus, since the S/N (Signal to Noise) ratio is poor, the results of computation obtained at the respective portions become not identical to each other in sign.

The computing unit 6 calculates each computed value of a computational equation using at least two parameters of three parameters: the strain obtained by the physical quantity data generator 5, the correlation coefficient related to the correlation computation, and the intensity of each echo signal of ultrasound obtained from the biological tissue (computing function). The details thereof will be described later.

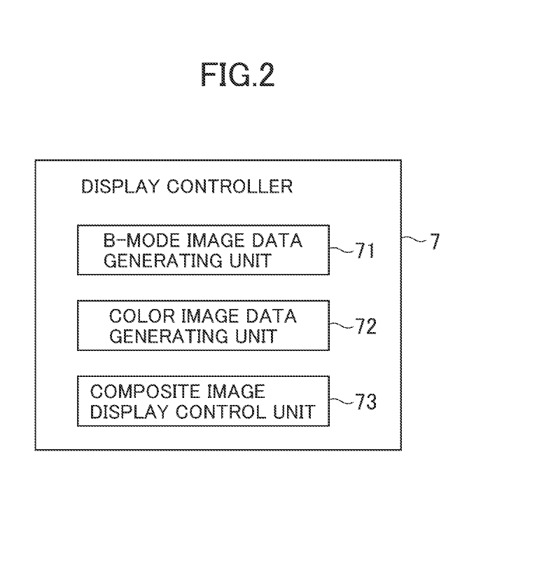

The display controller 7 is inputted with the B-mode data from the B-mode data generator 4 and data about the computed value obtained from the computing unit 6. As shown in FIG. 2, the display controller 7 has a B-mode image data generating unit 71, a color image data generating unit 72 and a composite image display control unit 73.

The B-mode image data generating unit 71 converts the B-mode data obtained by the B-mode data generator 4 to B-mode image data having information corresponding to the brightness that depends on the intensity of each echo signal. The color image data generating unit 72 converts the data of the computed value obtained by the computing unit 6 to information corresponding to the color to thereby generate color image data having information corresponding to the color that depends on the computed value. The color image data generating unit 72 brings the data of each computed value to gradation and generates color image data comprised of information corresponding to colors assigned to respective levels of grays. The number of gradations is 256, for example.

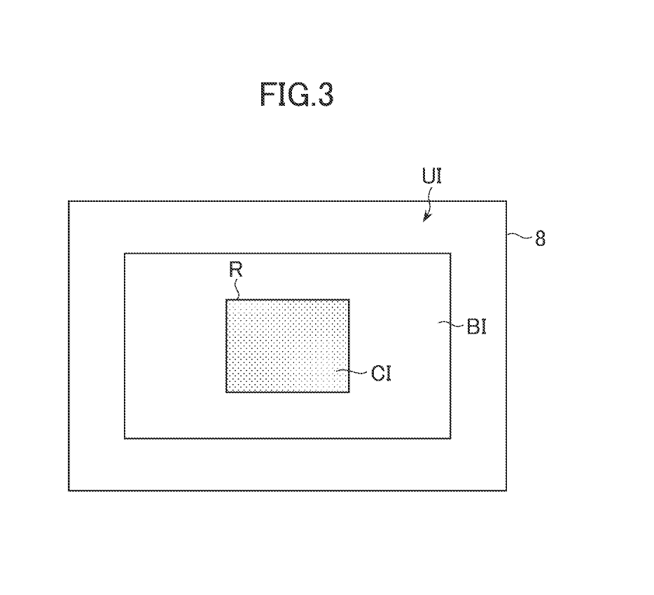

The composite image display control unit 73 combines the B-mode image data and the color image data together to generate image data about a composite ultrasound image displayed on the display unit 8. The composite image display control unit 73 causes the display unit 8 to display the image data thereof as a composite ultrasound image UI in which a B-mode image BI and a color image CI are combined together as shown in FIG. 3. The color image CI is displayed within a region R set to the B-mode image BI. The color image CI is displayed in semitransparent form (in a see-through state of B mode image corresponding to the background).

The display unit 8 includes, for example, an LCD (Liquid Crystal Display), a CRT (Cathode Ray Tube) or the like. The operation unit 9 includes a keyboard and a pointing device or the like (not shown) for inputting instructions and information by an operator.

The controller 10 has a CPU (Central Processing Unit) and reads a control program stored in the storage unit 11 to execute functions at the respective parts of the ultrasound diagnostic apparatus 1, starting with the physical quantity calculating function, the computing function, etc.

A description will now be made of the operation of the ultrasound diagnostic apparatus 1 according to the first embodiment. The ultrasound diagnostic apparatus 1 displays an image in which a cyst that is an object to be extracted in the first embodiment has been extracted from a biological tissue. This will be described specifically. The transmission/reception of ultrasound to and from the biological tissue is performed in a state in which the ultrasound probe 2 has been brought into contact with a body surface of a subject, thereby acquiring echo signals. When the transmission/reception of the ultrasound is performed, pressure to the biological tissue by the ultrasound probe 2 and its relaxation are performed.

The transmission/reception of the ultrasound is divided into two types. The transmission/reception of the ultrasound, which is used for a B-mode image, and the transmission/reception of the ultrasound, for calculating strain by the physical quantity data generator 5 are performed at their corresponding transmission/reception parameters.

When the echo signals are acquired, the B-mode data generator 4 generates the B-mode data. The physical quantity data generator 5 performs a correlation computation to calculate each strain of the biological tissue and generates the physical quantity data. The physical quantity data generator 5 performs a correlation computation on a plurality of points on one sound ray and thereby calculates strains at the respective points.

When the physical quantity data is obtained, the computing unit 6 performs an arithmetic computation using the following Equation 1:

F(n)=P(n)×Q(n) Equation 1

In the above Equation 1, n indicate whole numbers from 1 to N indicative of points (portions corresponding to correlation windows) on each sound ray where strains are calculated. P (n) is a function related to the strain, and Q(n) is a function related to a correlation coefficient at a correlation computation. Specifically, P (n) and Q (n) are given as the following by Equation 2 and Equation 3:

P(n)=k1×Abs(strain(n)) Equation 2

Q(n)=k2×(1/xCorr(n)) Equation 3

In Equation 2 and Equation 3, k1 and k2 are arbitrarily-set weighting factors. These k1 and k2 may be set as default values. Alternatively, they may be configured to be settable arbitrarily by an operator. strain (n) indicates the value of strain at each of the points 1 to N on the sound ray. Abs (strain (n)) is a function that returns the absolute value of the strain. Further, xCorr (n) is a value of a correlation coefficient at each of the points 1 to N on the sound ray.

F (n) serves as a function capable of extracting a cyst. This will specifically be explained. Assume that upon pressure, for example, as strains, a value of “−0.1%” is obtained in the cyst, a value of “+0.01%” is obtained in a tumor, and values of “+0.1%” and “+0.3%” are obtained at portions other than those. Assume that upon relaxation, values of opposite sign taken as strains, i.e., a value of “+0.1%” is obtained in the cyst, a value of “−0.01%” is obtained in the tumor, and values of “−0.1%” and “−0.3%” are obtained at portions other than those.

When the values of the strains such as described above are obtained, the strain of the cyst becomes a value indicative of it being harder than other portions. Therefore, when an elastic image is generated based on the value of the strain as in the related art, the cyst is represented in a color indicative of being hard. In the above Equation 2, however, the absolute value of the strain is obtained. Accordingly, the value of P (n) becomes a value indicating that the cyst becomes larger than the tumor and softer than the tumor. The cyst is smaller in correlation coefficient than other portions. Thus, the value of Q (n) obtained by the above Equation 3 also becomes large. In terms of the above, in the cyst, the value of F (n) becomes large, and F (n) becomes a function in which a computed value emphasized in parameter characteristics seen in the cyst and distinguishable from other than the cyst is obtained.

The computed value of F (n) obtained by the computing unit 6 is inputted to the display controller 7. The color image data generating unit 72 generates color image data having information corresponding to each color that depends on the computed value of F (n).

The B-mode image data generating unit 71 generates B-mode image data, based on the B-mode data generated by the B-mode data generator 4.

When the B-mode image data and the color image data are generated, the composite image display control unit 73 generates image data of a composite ultrasound image, based on these B-mode image data and color image data and allows the display unit 8 to display a composite ultrasound image UI as shown in FIG. 3.

The composite ultrasound image is an image in which a B-mode image BI and a color image CI are combined together. The color image CI is an image having each color corresponding to the computed value of F (n) and has colors such as blue, green, red, etc. For example, a portion in which the computed value of F (n) is small is represented in blue in the color image CI, whereas a portion in which the computed value of F (n) is large is represented in red in the color image CI. In this case, since the computed value of F (n) is large in the cyst, it is represented in red in the color image CI. Thus, according to the ultrasound diagnostic apparatus 1 of the first embodiment, an image from which a cyst has been extracted can be displayed.

On the other hand, in the tumor, the absolute value of strain is small, and the correlation coefficient is not necessarily small. Therefore, since the computed value of F(n) becomes smaller than the cyst in the tumor, the tumor is represented in a color different from one for the cyst in the color image CI. It is thus possible to distinguish between the tumor and the cyst in the color image CI.

Incidentally, although such P (n) and Q (n) that the computed value of F (n) becomes large are taken in the cyst in the above description, such P (n) and Q (n) that the computed value of F (n) becomes small may be taken in the cyst. Specifically, P (n) and Q (n) may be defined by Equation 2′ and Equation 3′:

P(n)=k1×{1/Abs(strain(n)) Equation 2′

Q(n)=k2×xCorr(n) Equation 3′

When P (n) and Q (n) are given as in the above Equation 2′ and Equation 3′, respectively, the cyst is represented in blue in the color image CI. On the other hand, since the computed value of F (n) in the tumor becomes larger than in the cyst, the tumor is represented in a color different from one for the cyst in the color image CI.

Modifications of the first embodiment will next be explained. A first modification will first be described. In the present example, the computing unit 6 performs an arithmetic operation or computation using the following Equation 11:

F(n)=P(n)×Q(n)×R(n) Equation 11

In the above Equation 11, P (n) and Q (n) correspond to the above Equation 2 and Equation 3 respectively. R (n) is given by the following Equation 12:

R(n)=k3×(1/Intensity(n)) Equation 12

In the Equation 12, k3 is an arbitrarily-set weighting factor. k3 may be set in default. Alternatively, k3 may be configured to be settable arbitrarily by an operator. Intensity (n) is the intensity of each of echo signals at the points 1 to N on the sound ray. The intensity of each echo signal may be an intensity of an echo signal obtained by the transmission/reception of ultrasound, which is used for the B-mode image, or may be an intensity of an echo signal obtained by the transmission/reception of ultrasound, for calculating strain by the physical quantity data generator 5.

Since the intensity of the echo signal is small in the cyst here, R (n) becomes a large value. Thus, the computed value of F (n) becomes large in the cyst.

In the above Equation 11, P (n) and Q (n) may be taken as in the above Equation 2′ and Equation 3′, respectively. In this case, R (n) is given by the following Equation 12′:

R(n)=k3×Intensity(n) Equation 12′
In this case, the computed value of F (n) becomes small in the cyst.

Even in the present example described above, since the computed value of F (n) becomes either large or small in the cyst, it is possible to display an image from which the cyst has been extracted.

A second modification will next be explained. In the present example, the computing unit 6 performs an arithmetic computation using the following Equation 21:

F(n)=P(n)×R(n) Equation 21

In the above Equation 21, P (n) and R (n) correspond to the above Equation 2 and Equation 12, respectively. In this case, the computed value of F (n) becomes large in the cyst. In the above Equation 21, P (n) and R (n) may be the above Equation 2′ and Equation 12′ respectively. In this case, the computed value of F (n) becomes small in the cyst.

Even in the present example described above, since the computed value of F (n) becomes either large or small in the cyst, it is possible to display an image from which the cyst has been extracted.

A third modification will next be explained. In the present example, the computing unit 6 performs an arithmetic computation using the following Equation 31:

F(n)=Q(n)×R(n) Equation 31

In the above Equation 31, Q (n) and R (n) correspond to the above Equation 3 and Equation 12, respectively. In this case, the computed value of F (n) becomes large in the cyst. In the above Equation 31, Q (n) and R (n) may be the above Equation 3′ and Equation 12′ respectively. In this case, the computed value of F (n) becomes small in the cyst.

Even in the present example described above, since the computed value of F (n) becomes either large or small in the cyst, it is possible to display an image from which the cyst has been extracted.

### Second Embodiment

A second embodiment will next be explained. Incidentally, items different from those in the first embodiment will be explained in the following description.

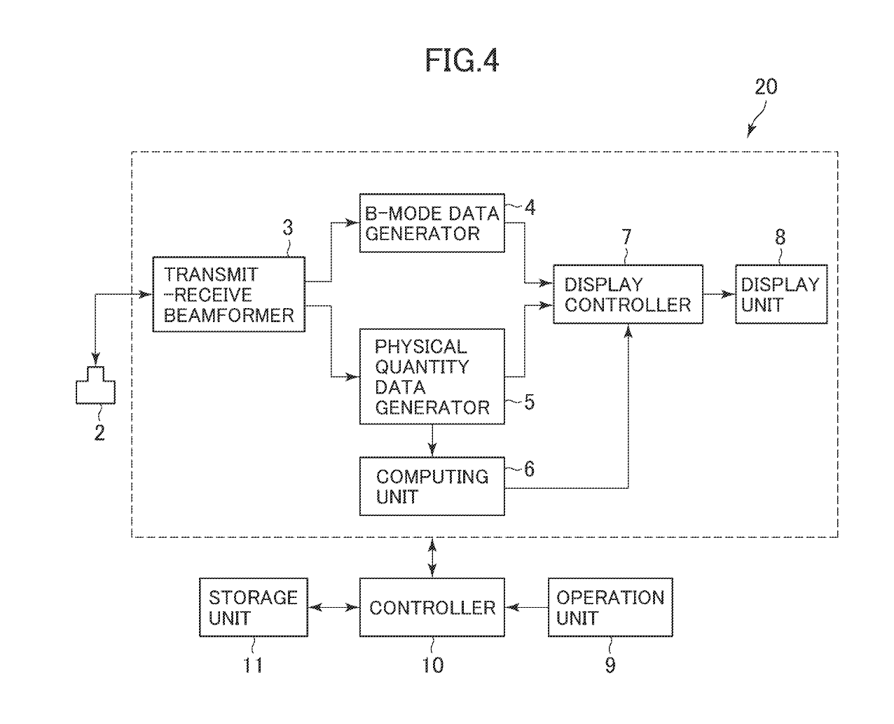

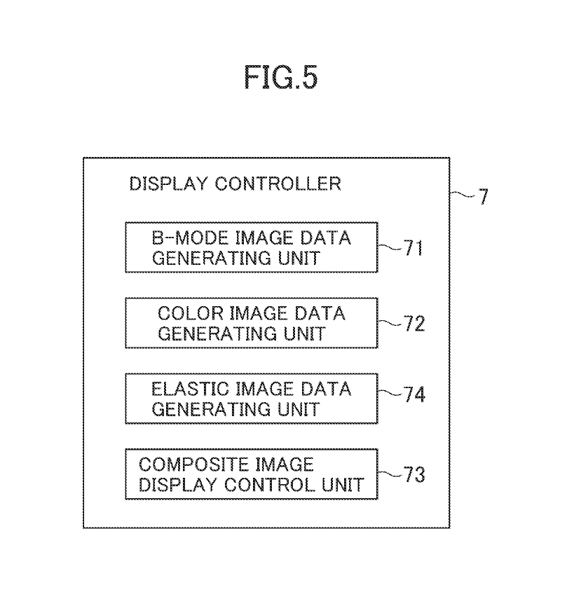

An ultrasound diagnostic apparatus 20 of the second embodiment shown in FIG. 4 is equipped with an ultrasound probe 2, a transmit-receive beamformer 3, a B-mode data generator 4, a physical quantity data generator 5, a computing unit 6, a display controller 7, a display unit 8, an operation unit 9, a controller 10 and a storage unit 11 as with the first embodiment. In the ultrasound diagnostic apparatus 20 of the second embodiment, however, physical quantity data generated by the physical quantity data generator 5 is inputted not only to the computing unit 6 but also to the display controller 7. As shown in FIG. 5, the display controller 7 has an elastic image data generating unit 74 in addition to a B-mode image data generating unit 71, a color image data generating unit 72 and a composite image display control unit 73. The elastic image data generating unit 74 converts the physical quantity data to information corresponding to each color and thereby generates elastic image data having information corresponding to each color that depends on the strain. The elastic image data generating unit 74 brings the data of the computed values to gradation and generates elastic image data comprised of information corresponding to colors assigned to respective levels of grays. The number of gradations is 256, for example. Variations in the colors of an elastic image displayed based on the elastic image data may be the same as the color image CI. When the strain is small, for example, the variations may be represented in blue. When the strain is large, the variations may be represented in red.

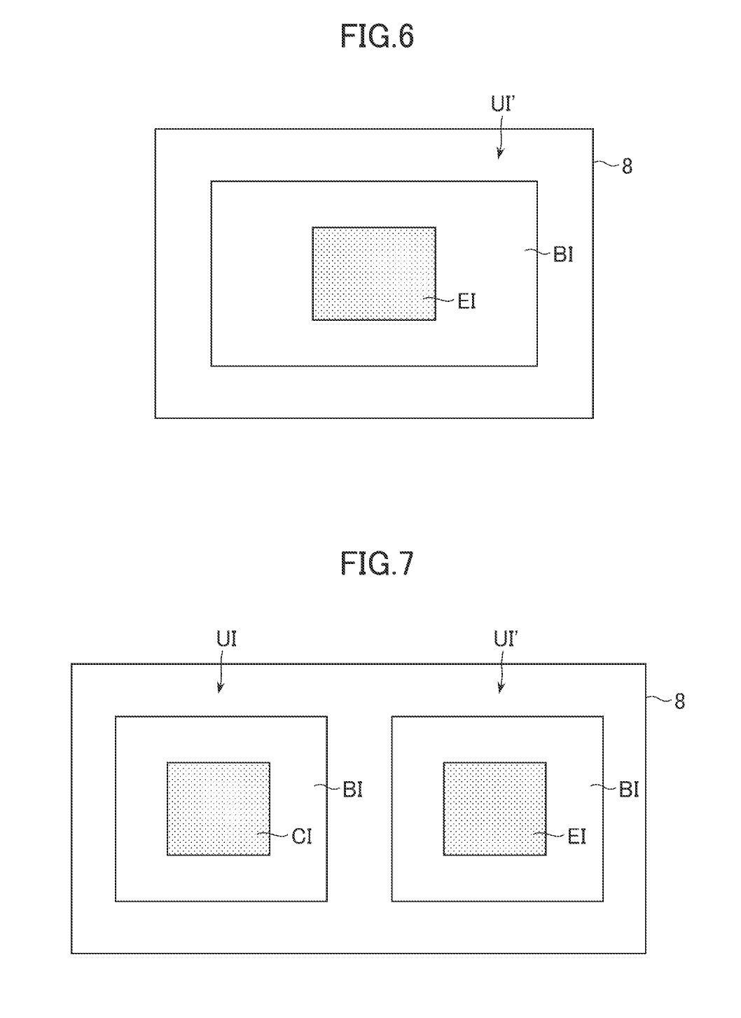

The operation of the second embodiment will be explained. In the ultrasound diagnostic apparatus 20 of the second embodiment, the composite image display control unit 73 causes the display unit 8 to display by switching between, a composite ultrasound image UI in which a B-mode image BI and a color image CI are combined together as shown in FIG. 3 and a composite ultrasound image UI′ in which a B-mode image BI and an elastic image EI are combined together as shown in FIG. 6. The composite image display control unit 73 may switch and display the composite ultrasound image UI and the composite ultrasound image UI′, based on an input by an operator to the operation unit 9.

The composite image display control unit 73 may cause the display unit 8 to display the composite ultrasound image UI and the composite ultrasound image UI′ side by side as shown in FIG. 7.

According to the ultrasound diagnostic apparatus 20 of the second embodiment, advantages similar to those in the first embodiment can be obtained. In addition to the above, a cyst and a tumor can more reliably be determined by allowing the operator to display the composite ultrasound image UI and the composite ultrasound image UI′ by switching or display them side by side.

Although the disclosure has been explained by the exemplary embodiments as described above, it is needless to say that the systems and method described herein can be changed in various ways within the scope of the invention that does not change the spirit of the invention.

Many widely different embodiments may be configured without departing from the spirit and the scope of the present invention. It should be understood that the disclosure is not limited to the specific embodiments described in the specification, except as defined in the appended claims.

The systems and methods described herein relate to an ultrasound diagnostic apparatus which emphasizes a computed value in parameter characteristics of a cyst in biological tissues, and the apparatus can display an image in which a tumor and the cyst can be distinguished each other.

1. An ultrasound diagnostic apparatus comprising:
a physical quantity calculating unit configured to:
set correlation windows to two echo signals different in time from each other on the same sound ray, the echo signals obtained by transmission/reception of ultrasound to and from a biological tissue; and
perform a correlation computation between the correlation windows to calculate a physical quantity related to elasticity of each portion in the biological tissue;
a computing unit configured to compute a computed value from a computational equation, the computed value emphasizing parameter characteristics of a cyst in the biological tissue and and distinguishing the cyst from other tissue in the biological tissue, the computational equation using at least two of the following three parameters: an absolute value of the physical quantity calculated by the physical quantity calculating unit, a correlation coefficient related to the correlation computation, and an intensity of each echo signal of ultrasound obtained from the biological tissue; and
a display unit configured to display an image having a display form corresponding to the computed value.

2. The ultrasound diagnostic apparatus according to claim 1, wherein the computational equation is the following equation:
F(n)=P(n)×Q(n);
wherein P (n)=k1×Abs (strain (n)) and Q (n)=k2×(1/xCorr (n)), or P (n)=k1×{1/abs (strain (n)) and Q (n)=k2×xCorr (n); and
wherein n are whole numbers from 1 to N indicative of points on a sound ray where strains are calculated by the physical quantity calculating unit, k1 and k2 are weighting factors, strain (n) are values of the strains at the points 1 to N on the sound ray, which have been calculated by the physical quantity calculating unit, Abs (strain (n)) is a function that returns the absolute value of each strain calculated by the physical quantity calculating unit, and xCorr (n) are values of correlation coefficients at the points 1 to N on the sound ray.

3. The ultrasound diagnostic apparatus according to claim 1, wherein the computational equation is the following equation:
F(n)=P(n)×Q(n)×R(n);
wherein P (n)=k1×Abs (strain (n)), Q (n)=k2×(1/xCorr (n)), and R (n)=k3×(1/Intensity (n)), or P (n)=k1×{1/abs (strain (n)), Q (n)=k2×xCorr (n), and R (n)=k3×Intensity (n); and
wherein n are whole numbers from 1 to N indicative of points on a sound ray where strains are calculated by the physical quantity calculating unit, k1, k2 and k3 are weighting factors, strain (n) are values of the strains at the points 1 to N on the sound ray, which have been calculated by the physical quantity calculating unit, Abs (strain (n)) is a function that returns the absolute value of each strain calculated by the physical quantity calculating unit, xCorr (n) are values of correlation coefficients at the points 1 to N on the sound ray of ultrasound, and Intensity (n) is an intensity of each of echo signals at the points 1 to N on the sound ray of ultrasound.

4. The ultrasound diagnostic apparatus according to claim 1, wherein the computational equation is the following equation:
F(n)=P(n)×R(n);
wherein P (n)=k1×Abs (strain (n)) and R (n)=k3×(1/Intensity (n)), or P (n)=k1×{1/abs (strain (n)) and R (n)=k3×Intensity (n): and wherein n are whole numbers from 1 to N indicative of points on a sound ray where strains are calculated by the physical quantity calculating unit, k1 and k3 are weighting factors, strain (n) are values of the strains at the points 1 to N on the sound ray, which have been calculated by the physical quantity calculating unit, Abs (strain (n)) is a function that returns the absolute value of each strain calculated by the physical quantity calculating unit, and Intensity (n) is an intensity of each of echo signals at the points 1 to N on the sound ray of ultrasound.

5. The ultrasound diagnostic apparatus according to claim 1, wherein the computational equation is the following equation:
F(n)=Q(n)×R(n)R(n);
wherein Q (n)=k2×(1/xCorr (n)) and R (n)=k3×(1/Intensity (n)), or Q (n)=k2×xCorr (n) and R (n)=k3×Intensity (n); and
wherein n are whole numbers from 1 to N indicative of points on a sound ray where strains are calculated by the physical quantity calculating unit, k1 and k3 are weighting factors, xCorr (n) are values of correlation coefficients at the points 1 to N on the sound ray of ultrasound, and Intensity (n) is an intensity of each of echo signals at the points 1 to N on the sound ray of ultrasound.

6. The ultrasound diagnostic apparatus according to claim 1, including an image display control unit configured to cause the display unit to display an image having a display form corresponding to the computed value of the computing unit, and an elastic image having a display form corresponding to the physical quantity by switching.

7. The ultrasound diagnostic apparatus according to claim 2, including an image display control unit configured to cause the display unit to display an image having a display form corresponding to the computed value of the computing unit, and an elastic image having a display form corresponding to the physical quantity by switching.

8. The ultrasound diagnostic apparatus according to claim 3, including an image display control unit configured to cause the display unit to display an image having a display form corresponding to the computed value of the computing unit, and an elastic image having a display form corresponding to the physical quantity by switching.

9. The ultrasound diagnostic apparatus according to claim 4, including an image display control unit configured to cause the display unit to display an image having a display form corresponding to the computed value of the computing unit, and an elastic image having a display form corresponding to the physical quantity by switching.

10. The ultrasound diagnostic apparatus according to claim 5, including an image display control unit configured to cause the display unit to display an image having a display form corresponding to the computed value of the computing unit, and an elastic image having a display form corresponding to the physical quantity by switching.

11. The ultrasound diagnostic apparatus according to claim 1, wherein the physical quantity calculating unit is configured to compute a complex cross-correlation imaginary part between the correlation windows to calculate a strain of the biological tissue.

12. The ultrasound diagnostic apparatus according to claim 2, wherein the physical quantity calculating unit is configured to compute a complex cross-correlation imaginary part between the correlation windows to calculate a strain of the biological tissue.

13. The ultrasound diagnostic apparatus according to claim 3, wherein the physical quantity calculating unit is configured to compute a complex cross-correlation imaginary part between the correlation windows to calculate a strain of the biological tissue.

14. The ultrasound diagnostic apparatus according to claim 4, wherein the physical quantity calculating unit is configured to compute a complex cross-correlation imaginary part between the correlation windows to calculate a strain of the biological tissue.

15. The ultrasound diagnostic apparatus according to claim 5, wherein the physical quantity calculating unit is configured to compute a complex cross-correlation imaginary part between the correlation windows to calculate a strain of the biological tissue.

16. The ultrasound diagnostic apparatus according to claim 6, wherein the physical quantity calculating unit is configured to compute a complex cross-correlation imaginary part between the correlation windows to calculate a strain of the biological tissue.

17. The ultrasound diagnostic apparatus according to claim 7, wherein the physical quantity calculating unit is configured to compute a complex cross-correlation imaginary part between the correlation windows to calculate a strain of the biological tissue.

18. The ultrasound diagnostic apparatus according to claim 8, wherein the physical quantity calculating unit is configured to compute a complex cross-correlation imaginary part between the correlation windows to calculate a strain of the biological tissue.

19. The ultrasound diagnostic apparatus according to claim 9, wherein the physical quantity calculating unit is configured to compute a complex cross-correlation imaginary part between the correlation windows to calculate a strain of the biological tissue.

20. A method for displaying an ultrasound image, the method comprising:
setting correlation windows to two echo signals different in time from each other on the same sound ray, the echo signals obtained by transmission/reception of ultrasound to and from a biological tissue;
performing a correlation computation between the correlation windows to calculate a physical quantity related to elasticity of each portion in the biological tissue;
computing a computed value from a computational equation, the computed value emphasizing parameter characteristics of a cyst in the biological tissue and distinguishing the cyst from other tissue in the biological tissue, the computational equation using at least two of the following three parameters: an absolute value of the physical quantity, a correlation coefficient related to the correlation computation, and an intensity of each echo signal of ultrasound obtained from the biological tissue; and
displaying an image having a display form corresponding to the computed value.
